(19) United States
(12) Patent Application Publication
(10) Pub. No.: US 2010/0324592 A1
(43) Pub. Date: Dec. 23, 2010

(54) SUTURE-TYING FORCEPS

(76) Inventor: Sunho Kim, Seoul (KR)

Correspondence Address: OHLANDT, GREELEY, RUGGIERO & PERLE, LLP, ONE LANDMARK SQUARE, 10TH FLOOR, STAMFORD, CT, 06901, US

(21) Appl. No.: 12/870,231

(22) Filed: Aug. 27, 2010

(30) Foreign Application Priority Data
Mar. 4, 2008 (KR) 10-2008-0020064
Feb. 27, 2009 (KR) PCT/KR2009/000969

(51) Int. Cl. A61B 17/28

(52) U.S. Cl. 606/205

(57) ABSTRACT

A suture-tying forceps which are generally used in surgical operations. The suture-tying forceps comprises a forceps part, a body part and an adjusting part, with the forceps part consisting of an upper jaw part and a lower jaw part which have two through holes being connected through an internal tunnel and which are engaged with each other. The suture-tying forceps results in a successful watertight dural closure since it is possible to reliably tie a suture in an operation which is generally performed in a narrow, deep surgical field such as that in TSA, and can be widely applied to another surgical fields with similar situation which had difficulty of needle handling work like spinal surgery.

### BACKGROUND

1. Field of the Disclosure

The present disclosure relates to a suture-tying forceps, and in particular to a suture-tying forceps which consists of a forceps part, an adjusting part and a body part.

2. Discussion of the Background Art

One of the most common postoperative complications after transsphenoidal approach (TSA) is a cerebrospinal fluid (CSF) leak that typically results from inadequate repair of a CSF fistula created at the time of the initial operation. Most techniques utilize autologous tissue grafts of fat, muscle or fascia lata, with or without the use of postoperative lumbar CSF drainage.

In recent years, the extended transsphenoidal approach has been applied in case of large suprasellar and parasellar tumors. The extended TSA provides a direct access to the interior surface of the chiasm or third ventricle, without retraction of the brain. However, the most serious complication arising from this approach is CSF rhinorrhea. So, a watertight reconstruction is fundamental in preventing the complications related to a postoperative CSF leakage.

Although watertight suturing of the dura mater is desirable for prevention of CSF leakage, it is technically difficult in a narrow, deep surgical field such as that in TSA. Some special techniques and instruments for direct suturing of the dura mater during TSA have been described; because these techniques are complicated and impractical, however, they have not been widely accepted.

Accordingly, it is an object of the present disclosure to provide a suture-tying forceps which results in a successful watertight dural closure at the sellar floor level to prevent postoperative CSF leakage.

### SUMMARY

To achieve the above objects, there is provided a suture-tying forceps which comprises a forceps part which includes an upper jaw and a lower jaw, an adjusting part and a body part.

A forceps part were consisted with two, upper and lower jaws which engaged with each other. A hole was located at the tip of each jaw and another hole was located on the dorsum of the each jaw. These two holes on each jaw were connected with tunnel. A needle or a thread pass through the holes located at the tip of the jaw and pass out from the hole located at the dorsum of the jaw. An adjusting part is connected with the forceps part by means of a connection means which operates the forceps part.

And the body part includes a driving part which connects the connection means and the adjusting part. This driving part slides by means of an operation of the adjusting part and thereby rotates the forceps part around the connection means as an axis. And the body part includes a support part on which the driving part slides.

According to the present disclosure, the suture-tying forceps would result in a successful watertight dural closure since it is possible to reliably tie a suture in an operation which is generally performed in a narrow, deep surgical field such as that in TSA. In addition, the present disclosure would be widely applied to another surgical fields with similar situation which had difficulty of needle handling work like spinal surgery.

### BRIEF DESCRIPTION OF THE DRAWINGS

The present disclosure will become better understood with reference to the accompanying drawings which are given only by way of illustration and thus are not limitative of the present disclosure, wherein;

FIG. 1 is a schematic perspective view illustrating a suture-tying forceps according to an embodiment of the present disclosure;

FIG. 2 is a disassembled perspective view illustrating a forceps part and an adjusting part of a suture-tying forceps of FIG. 1;

FIG. 3 is an assembled perspective view illustrating a forceps part and an adjusting part of a suture-tying forceps of FIG. 2;

FIG. 4 is a schematic cross sectional view illustrating an operation procedure of a suture-tying forceps of FIG. 1;

FIG. 5 is a plane view illustrating a suture-tying forceps according to another embodiment of the present disclosure;

FIG. 6 is a disassembled perspective view illustrating a forceps part and an adjusting part of a suture-tying forceps of FIG. 5;

FIG. 7 is an assembled perspective view illustrating a forceps part and an adjusting part engaged in a suture-tying forceps of FIG. 7;

FIG. 8 is a disassembled perspective view illustrating a forceps part and an adjusting part of a suture-tying forceps according to further another embodiment of the present disclosure;

FIG. 9 is an assembled perspective view illustrating a forceps part and an adjusting part engaged in a suture-tying forceps of FIG. 8; and

FIG. 10 is a schematic view for describing a dura mater suturing procedure using a suture-tying forceps after a TSA is performed according to the present disclosure.

### DETAILED DESCRIPTION OF THE PREFERRED EMBODIMENT

In the present disclosure, a suture-tying forceps comprises a forceps part which includes an upper jaw and a lower jaw engaged with each other; an adjusting part which operates the forceps part; and a body part of which a proximal end is connected with the forceps part by means of a connection means, and a distal end is connected with the adjusting part, wherein a hole is formed in the upper jaw and the lower jaw, respectively, with a needle or a thread passing through the holes, wherein the hole is located at the tip of each jaw and another hole is located on the dorsum of the each jaw and the two holes on each jaw is connected with tunnel, wherein said body part includes a driving part which connects the connection means and the adjusting part and slides by means of an operation of the adjusting part for thereby rotating the forceps part around the connection means as an axis, and a support part on which the driving part slides.

The preferred embodiments of the present disclosure will be described with reference to the accompanying drawings.

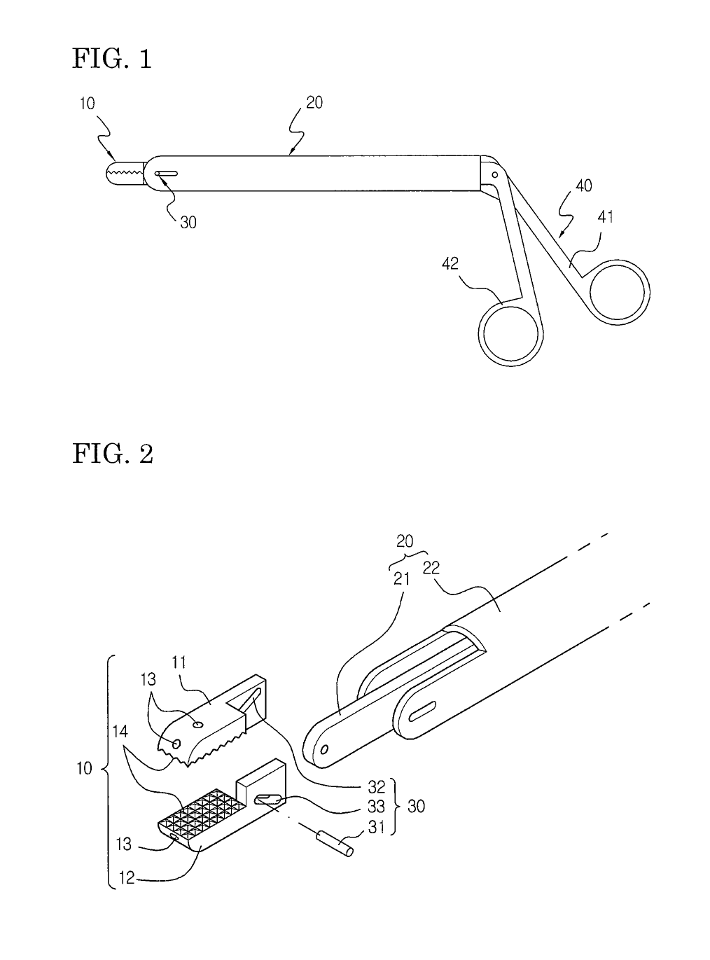

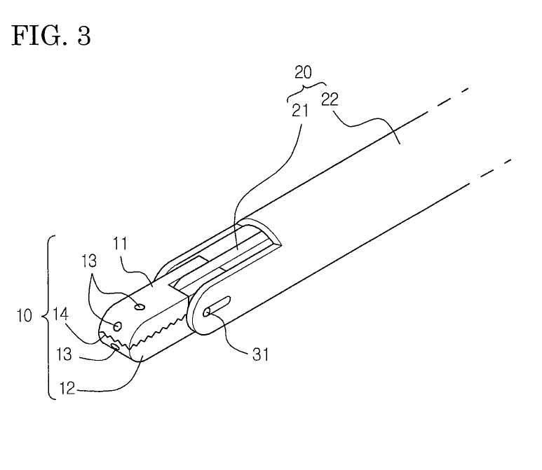

FIG. 1 is a schematic perspective view illustrating a suture-tying forceps according to an embodiment of the present disclosure, FIG. 2 is a disassembled perspective view illustrating a forceps part and a body part of a suture-tying forceps of FIG. 1, and FIG. 3 is an assembled perspective view illustrating a forceps part and a body part of a suture-tying forceps of FIG. 2.

As shown in FIG. 1, the present disclosure is basically directed to a suture-tying forceps which are generally used in surgical operations. The suture-tying forceps according to the present disclosure comprises a forceps part 10, a body part 20 and an adjusting part 40. The suture-tying forceps according to the present disclosure would be used in a dural suture tying work as it is inserted into a nasal speculum.

The forceps part 10 consists of an upper jaw 11 and a lower jaw 12. The upper and lower jaw 11 and 12 can be engaged with each other, and holes 13 and 13′ are formed in the upper and lower jaws 11 and 12, respectively. The hole was located at the tip of each jaw and another hole was located on the dorsum of the each jaw. These two holes on each jaw were connected with tunnel. A needle or a thread pass through the holes located at the tip of the jaw and pass out from the hole located at the dorsum of the jaw.

A needle or a thread passes through the through holes 13 and 13′ so their sizes are not limited. The holes are large enough to pass through a needle or a thread. The hole 13 is used for forming a knot by inserting a suture thread. So, a watertight closure can be easily formed using the suture-tying forceps according to the present disclosure.

A pair of needle holder tips 14, which are engaged with each other, would be provided in the upper and lower jaws 11 and 12, respectively. The needle holder tip 14 is configured to basically hold a needle from movements. It is obvious to those skilled in the art that the construction of the needle holder tip 14 is not limited. A certain structure, which can stably and reliably hold the needle, would be used. A toothed shape is most preferred. The forceps part 10 functions for fixing a needle in the course of wound suture, so a dural tissue can be reliably sewed in a nasal speculum.

The proximal end of the body part 20 is connected with the forceps part 10 by means of a connection means 30, and the distal end of the same is connected with the adjusting part 40.

The body part 20 is configured as substantially being thin and long for a work in a narrow and deep nasal speculum. It is obvious to those who skilled in the art that there is not any limit in the thickness of the body part 20 even if it is substantially long and thin for an insertion work in a nasal speculum. Preferably, the thickness of the body part 20 is less than 3 mm. The length of the body part 20 is determined so that a surgical operator can conveniently work outside the nasal speculum of a patient, which length is not limited thereto. The length of the same is preferably 13 through 15 cm.

The body part 20 consists of a driving part 21 and a support part 22. The driving part 21 connects the connection means 30 and the adjusting part 40 and slides by means of an operation of the adjusting part 40 for thereby rotating the forceps part 10 around the connection means 30 as an axis. The driving part 21 slides on the support part 22.

As shown in FIGS. 1 through 3, the support part 22 would be configured as being hollow. The driving part 21 would be positioned in the support part.

It is obvious to those who skilled in the art that the driving part 21 would be formed in certain shape or would be formed of a certain material if they can perform the above operations. The shape and material are not limited thereto. For example, the driving part 21 would be formed of a bar, wire, rope, thread, band, cord or cable.

The connection means 30 is configured to change a straight motion, which is caused as the driving part 21 slides, into a rotational motion for thereby driving the forceps part 10. Any device or apparatus can be used if it can operate the forceps 10 without limits.

As shown in FIG. 2, the connection part 30 according to an embodiment of the present disclosure includes a hinge shaft 31, and upper and lower small driving holes 32 and 33 for providing passages for the movement of the hinge shaft.

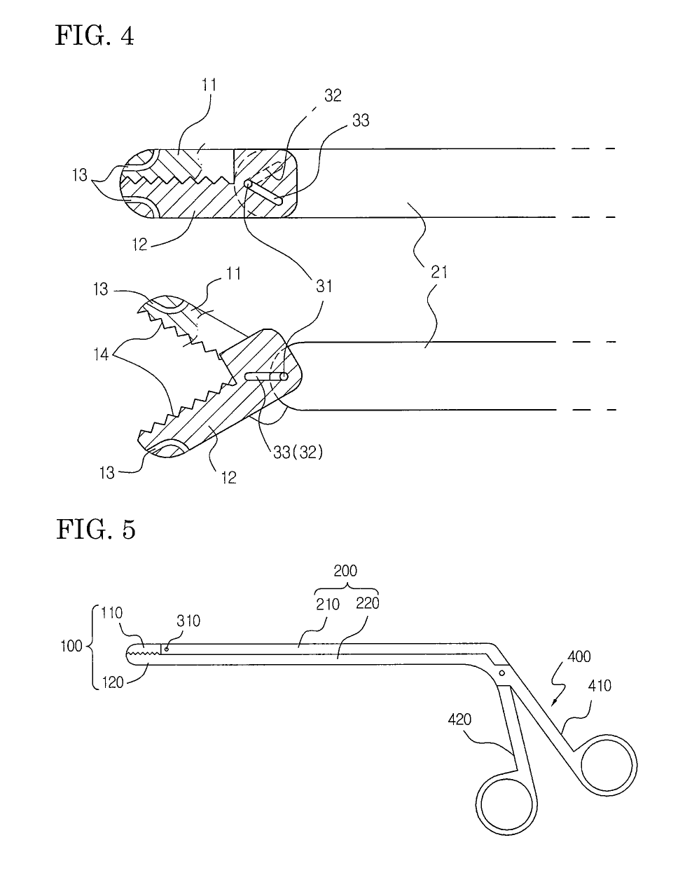

FIG. 4 is a schematic view illustrating an operation process of a suture-tying forceps of FIG. 1. As shown in FIG. 4, when the driving part 21 slides backwards, the hinge shaft 31 rotates the upper and lower jaws 11 and 12 and separates the same which were engaged as the hinge shaft 31 are moved along the upper and lower small holes 32 and 33.

When the driving part 21 slides forwards, the hinge shaft 31 moves along the upper and lower small holes 32 and 33 for thereby closely engaging the upper and lower jaws 11 and 12. However, the operation might be performed in a reverse direction by changing the position and structure of the small driving holes 32 and 33 and the hinge shaft.

The adjusting part 40 slides the driving part 21 of the body part for thereby operating the forceps part 10. Here, the adjusting part 40 includes a fixing adjusting part 41 formed at a remote end of the support part 22, and a driving adjusting part 42 formed at the remote end of the driving part 21. In another embodiment of the present disclosure, a driving adjusting part would be formed at a remote end of the support part 22, and a fixing adjusting part 42 would be formed at a remote end of the driving part 21.

As the driving adjusting part 42, any device or apparatus can be used even if it can slide the driving part 21. As shown in FIG. 1, a scissors-shaped adjusting part 40 is disclosed as an example. In the adjusting part of FIG. 1, the fixing adjusting part 41 is integrally formed at a remote end of the support part 22, and the driving adjusting part 42 is integrally formed in the driving part 21, and they are connected by means of the hinge shaft 43. When the driving adjusting part 42 moves backwards, the driving part 21 connected with the driving adjusting part gradually moves forwards for thereby driving the forceps 10. Here, as the driving adjusting part 42, a screw configured for changing the rotational motion into the straight motion can be used. A bar and spring can be used.

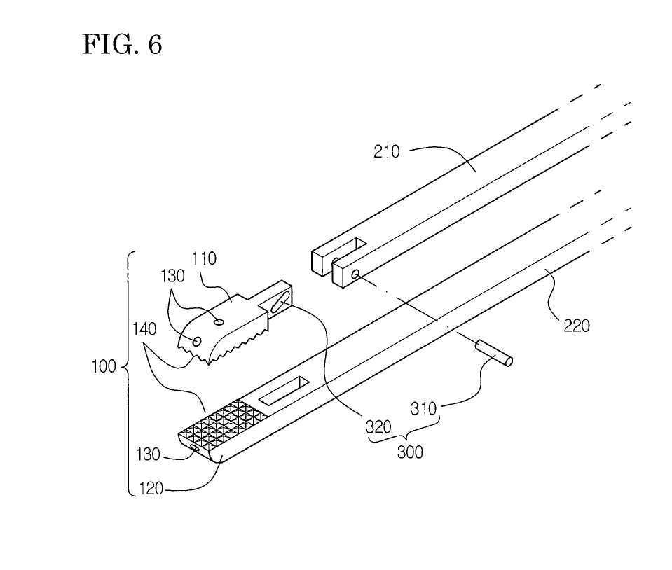

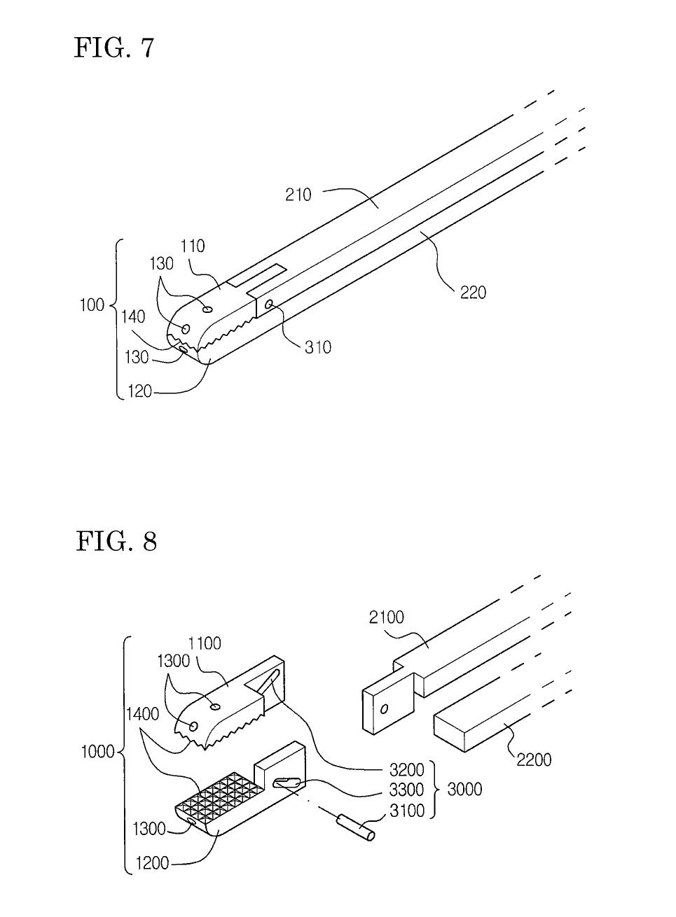

FIG. 5 is a plane view illustrating a suture-tying forceps according to another embodiment of the present disclosure. FIG. 6 is a disassembled perspective view illustrating a forceps part and a body part of a suture-tying forceps of FIG. 5. FIG. 7 is an assembled perspective view illustrating a forceps part and a body party engaged in a suture-tying forceps of FIG. 7.

As shown in FIGS. 5 through 7, the suture-tying forceps according to the present disclosure comprises a forceps part 100, a body part 200 and an adjusting part 400.

The forceps part 100 includes an upper jaw 110 and a lower jaw 120. The upper and lower jaws 110 and 120 are engaged with each other and include needle holder tips 140, respectively, which are engaged with each other as well. Holes 130 and 130′ are formed in the upper and lower forceps parts 110 and 120, respectively. The holes 130 and 130′ are formed passing through from one surface to the other surface where the upper and lower jaws are engaged with each other, which thus form an internal tunnel.

In the suture-tying forceps of FIGS. 5 through 7, a proximal end of the support part 220 is integrally connected with the lower jaw 120, and the driving part 210 is connected to the upper jaw 110 by means of the connection means 300. In the present disclosure, the support part 220 is formed not in a hollow cylindrical shape, but in a bar shape for thereby supporting the driving part 210 in its lower side.

The connection means 300 includes a hinge shaft 310, and an upper small driving hole 320.

In the above embodiment of the present disclosure, the driving part 210 slides by means of an operation of the adjusting part 400 for thereby rotating the upper jaw 110 with respect to the hinge shaft 310 as an axis. In the suture-tying forceps according to the present disclosure, the lower jaw 120 is fixed, whereas the upper jaw 110 operates.

In the more detailed operations, when the driving part 210 slides backwards, the hinge shaft 310 moves along the upper small driving hole 320 and rotates the upper forceps part 110, which was engaged, for thereby widening the upper forceps part 110.

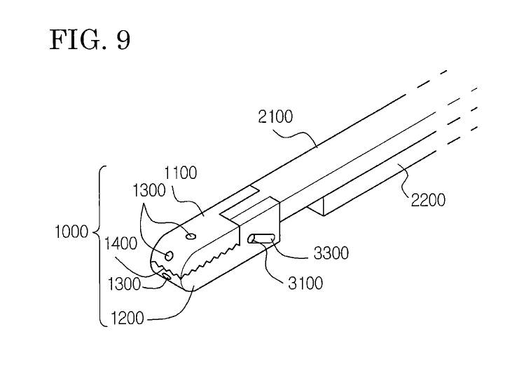

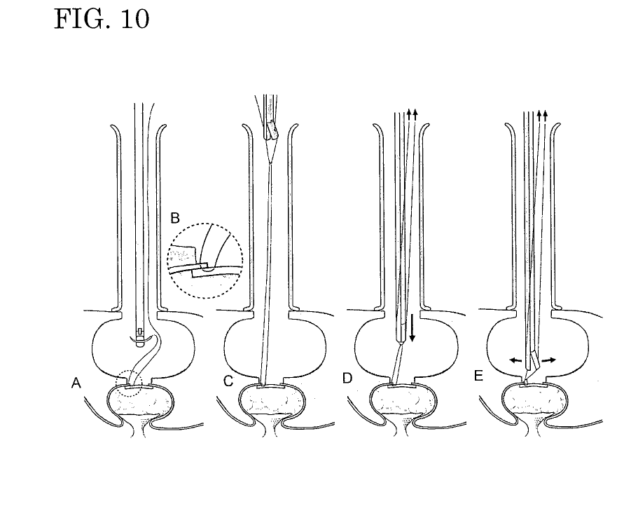

The operation would be performed in a reverse direction by changing the position and structure of the hinge shaft 310 and the small driving hole 320. FIG. 8 is a disassembled perspective view illustrating a forceps part and a body part of a suture-tying forceps according to further another embodiment of the present disclosure. FIG. 9 is an assembled perspective view illustrating a forceps part and a body part engaged in a suture-tying forceps of FIG. 8. The embodiment of FIGS. 8 and 9 is characterized in that the upper jaw 1100 and the lower jaw 1200 can be separately operable. Since the remaining structures and functions are same as the embodiments of FIGS. 5 through 7, so the detailed descriptions of the same will be omitted.

The operations of the suture-tying forceps according to the present disclosure will be described as follows.

FIG. 10 is a schematic view for describing a dura mater suturing procedure using a suture-tying forceps after a TSA is performed according to the present disclosure. Generally, the fascia obtained from femoral muscle is placed in an inner side for suturing dura mater and is stitched.

FIG. 10-A shows a step for a first stitch of fascia and dura mater while a needle is being supported by means of the forceps parts of the suture-tying forceps in a nasal speculum according to the present disclosure. The first stitch is placed on one of four corners of the fascia while it is placed inside of dural defect. At this point, the needle should not penetrate the whole thickness of facia, and about half thickness is enough (FIG. 10-B).

The forceps is pulled out of a nasal speculum, and needle retrieved, and a knob is made without applying a tensional force while holding both ends of the suture. The both ends of the suture are made to pass from the holes of the jaws of the upper and lower forceps parts 11 and 12 to the back or front holes, so that the suture knotted like FIG. 10-C is held by means of the forceps.

As shown in FIG. 10-D, the both ends of the suture are held by means of one hand with a proper tensional force, and the suture-tying forceps is made to advance by handling it with the other hand, so the knot advances into an operation portion of the nasal speculum.

As shown in FIG. 10-E, when the knot is placed at about 5 mm higher portion where the first stitch is made, the knot is adjusted to be placed at a desired position by repeatedly performing the opening and closing operations of the forceps while maintaining a certain tensional force in the suture by operating the upper forceps parts as force is applied to the adjusting part of the forceps. The tensional force of suture is released, and the forceps is pulled out of the nasal speculum, so a first knot formation is finished. A second knot and a third knot might be formed by repeatedly performing the above procedures.

The suture-tying forceps according to the present disclosure would be practically applied to a stitch formation procedure while fixing a needle with the helps of a forceps part and would be applied to a dura mater suturing procedure in such a manner that the both ends of suture are made to pass through the through holes formed in the forceps parts and are inserted into the nasal speculum for thereby forming a reliable knot at a desired position.

The suture-tying forceps according to the present disclosure makes it possible to easily make a knot of suture in a surgical operation which is performed in a narrow and deep portion like TSA, so a reliable watertight can be obtained. Since the needle can be easily handled using the forceps according to the present disclosure, this instrument would be widely applied to another surgical fields with similar situation which had difficulty of needle handling work like spinal surgery.

As described above, the suture-tying forceps according to the present disclosure would result in a successful watertight dural closure since it is possible to reliably tie a suture in an operation which is generally performed in a narrow, deep surgical field such as that in TSA. In addition, the present disclosure would be widely applied to surgical fields with its easier needle handling work with the helps of forceps.

As the present disclosure may be embodied in several forms without departing from the spirit or essential characteristics thereof, it should also be understood that the above-described examples are not limited by any of the details of the foregoing description, unless otherwise specified, but rather should be construed broadly within its spirit and scope as defined in the appended claims, and therefore all changes and modifications that fall within the meets and bounds of the claims, or equivalences of such meets and bounds are therefore intended to be embraced by the appended claims.

1. A suture-tying forceps, comprising:
a forceps part which includes an upper jaw and a lower jaw engaged with each other;
an adjusting part which operates the forceps part; and
a body part of which a proximal end is connected with the forceps part by means of a connection means, and a distal end is connected with the adjusting part,
wherein a hole is formed in the upper jaw and the lower jaw, respectively, with a needle or a thread passing through the holes, wherein the hole is located at the tip of each jaw and another hole is located on the dorsum of the each jaw and the two holes on each jaw is connected with tunnel,
wherein said body part includes a driving part which connects the connection means and the adjusting part and slides by means of an operation of the adjusting part for thereby rotating the forceps part around the connection means as an axis, and a support part on which the driving part slides.

2. The forceps of claim 1, wherein a proximal end of the support part is integrally connected with the lower jaw, and the driving part is connected with the upper jaw by means of the connection means, and the driving part slides by means of an operation of the adjusting part for thereby rotating the upper jaw with respect to the connection means as an axis.

3. The forceps of claim 1, wherein the interior of the support part is hollow, and said driving part is positioned in the interior of the support part.

4. The forceps of claim 1, wherein said driving part is positioned on an upper side of the support part.

5. The forceps of claim 1, wherein said connection means includes a hinge shaft, and a small driving hole which becomes a passage through which the hinge shaft moves.

6. The forceps of claim 1, wherein said adjusting part includes a fixing adjusting part formed at a remote end of the support part, and a driving adjusting part formed at a remote end of the driving part.

7. The forceps of claim 1, wherein said upper jaw and lower jaw are equipped with needle holder tips, respectively, which are engaged with each other.

8. The forceps of claim 1, wherein said driving part is selected from the group consisting of a bar, wire, rope, thread, cord and cable.
